(19) United States
(12) Patent Application Publication
(10) Pub. No.: US 2021/0107726 A1
(43) Pub. Date: Apr. 15, 2021

(54) Dispensing Box For Flexible Gloves

(71) Applicant: Wucon Tech KG, St. Egyden (AT)

(72) Inventor: Siegfried Wukovnig, St. Egyden am Steinfeld (AT)

(21) Appl. No.: 17/041,073

(22) PCT Filed: Mar. 25, 2019
(86) PCT No.: PCT/AT2019/060100

§ 371 Date: Sep. 24, 2020

(30) Foreign Application Priority Data
Mar. 25, 2018 (AT) A60047/2018

(51) Int. Cl. B65D 83/08; A61B 42/40; A61B 42/10

(52) U.S. Cl. CPC B65D 83/0805; A61B 42/10; A61B 42/40; B65D 83/0894

(57) ABSTRACT

A dispensing box for flexible gloves has a plurality of removal openings, which are positioned in such a way that, when the gloves are arranged parallel in the dispensing box according to the present teaching, the gloves can be grasped for removal only at the glove cuff, or at the cuff edge, or at a gripping aid positioned on the glove cuff, and that, when the removal opening in question is open, the palms and finger regions of the gloves in the dispensing box are covered by the dispensing box. The dispensing box preferably has two opposite removal openings. In order to separate the glove stacks in the dispensing box, the dispensing box has a separating film or a separating bag, the separating film or the separating bag covering, at least on one side, the finger and palm parts of the glove stacks, which glove stacks are oriented substantially parallel.

### TECHNICAL FIELD

The present teaching relates to a dispensing box for gloves, in particular work gloves or examination gloves which are intended for hygienic areas and are removed in non-sterile condition from the inventive dispensing box. The inventive dispensing box comprises one, or preferably two, removal openings which are positioned in such a way that the gloves can only be grasped deliberately by the cuff edge, thus avoiding external contamination of the unused gloves in the palm and finger regions in the dispensing box by users' soiled hands.

### BACKGROUND

At the present time in the USA, every tenth hospital patient is infected with a hospital infection, which is generally referred to as a nosocomial infection. The two million patients per year so affected cause the US health care system $4.5 to $11 billion in additional costs. Approximately 90,000 deaths are caused in the USA each year by these “hospital germs,” and it is estimated that at least 35% of these infections are easily avoidable. The situation in Europe is similar: Germany, for instance, reports more than 500,000 nosocomial infection cases each year, along with many thousands of deaths and additional costs of well over 2.5 billion euros.

The need to protect caregivers worldwide in the past two decades has led to an almost hundred-fold increase in the use of examination gloves. Simultaneously, however, and contrary to all expectations, there has been a great rise in nosocomial infections, including frequent cases even among hospital staff. This can be traced to the fact that, first, owing to the use of examination gloves made of latex or soft PVC, occlusive entrapment of human skin inside the glove leads to increased growth of bacteria and, secondly, it can be assumed that most caretakers coming into contact with patients and contaminated waste wear gloves, while the hands themselves are washed and disinfected less frequently.

A substantial number of nosocomial infections are transmitted by way of contaminated hands of hospital and caregiving personnel. An essential influence here is incorrect donning and removal of examination gloves along with inadequate hand hygiene. Germs are primarily deposited on the outside of unused gloves by caregivers' hands contaminated by preceding tasks, and then onto medical equipment and to the patients' surroundings.

Thus, in removing examination gloves, in fact there is always a critical contamination of gloves lying unused in the package or of gloves just recently put on. Subsequent infections of patients, for example in inserting catheters or infusions, are likely to occur, in particular owing to the transmission of germs and carriers of illness growing primarily on human tissue. The situation is worsened because in recent years gloves made of natural or synthetic latex tend to have fewer chemical deposits because of issues of compatibility with skin and thus there are scarcely any antibacterial rubber chemicals, such as dithiocarbamate accelerators, still present in the rubber film; this drastically increases the survival rate of bacteria on rubber surfaces. Owing to the constantly increasing use of powder-free examination gloves, no powder deposits remain on the hands of caregivers after gloves are removed and the result is a subjectively false impression of “clean, germ-free” hands within the gloves, which leads to an additional reduction in hand washing.

The non-sterile gloves used in the health care sector are usually delivered in non-sterile form in so-called dispensing box packages with a total count of 100 gloves usable for either hand. However, because of fully automated production facilities and GMP-certified production and packaging methods, prior contamination of gloves rarely occurs. Consequently, the sector has set the goal of breaking the chain of contamination from the germ source to the surface of the freshly put-on examination glove.

The most obvious means of interrupting the aforementioned contamination chain is continuous washing and disinfection of caregivers' hands. Essentially, however, this is a purely organizational measure, because, from the standpoint of skin compatibility, the use of disinfectant-containing inner glove surfaces under conditions of occlusive skin contact for extended wearing periods is not free of problems.

The present teaching is concerned here with avoiding contamination of unused, freshly put-on, non-sterile-packaged gloves, before they are bought into contact with patients or medical devices. In this process it is now common to encounter critical infections on the external surface of the gloves upon removal from the dispensing box and when the gloves are put on.

In addition, it is proposed with the present teaching that the inventive dispensing box should be filled with disposable gloves, which contain surfaces that act as disinfectants.

The following text considers a few of the many patents for dispensing boxes for gloves. All embodiments known to date, however, comprise only a single removal opening, and removal of the gloves leads to contamination of the gloves' exteriors in the critical region of the palms and/or fingers. In addition, many of the published patents are concerned with dispensing apparatuses, into which the gloves being used are not inserted until immediately before use by the user.

Patent U.S. Pat. No. 5,875,962 describes a dispensing box for gloves that is made of cardboard and comprises one closable removal opening.

Patent WO 9948782 describes a firmly mounted dispensing device for removing thin disposable gloves.

Patent US 20030057222 describes a typical dispensing box for repeat use with stacked gloves, such that the point of removal is situated in the center of the covering surface of the dispensing box in the area of the palms of the gloves.

The present state of the art in packing technology for flexible, non-sterile gloves such as examination gloves is to insert them, staggered in parallel, in the dispensing box. As a result of the parallel orientation, the gloves can be distributed uniformly over the predominantly brick-shaped package and small dispensing box sizes are sufficient so that packaging and transport costs are kept low. In addition, this arrangement ensures that the gloves can easily be removed individually without causing a number of additional gloves to be pulled out of the package during removal from the dispensing box. The dispensing boxes are made primarily of cardboard and comprise a removal opening, which is formed by tearing out a perforated area on the upper side of the dispensing box. The gloves are usually arranged parallel to the normally lengthwise removal opening and are clutched during removal from the outside in the palm region and pulled through this opening. The result, in practice, in every removal is contamination of the exterior of the glove by the germ-infested hands of the person removing it.

Proceeding from this fact, patent US 2007215628 A1, in turn, describes a dispensing box with two removal openings on the periphery of the case, so that the hygienically critical areas of the finger and palm of the gloves remain covered even when the dispensing box is open. As a result, both glove stacks situated in the dispensing box are placed at a 180° angle and are separated from one another by fixed liner bases. The liner bases are coupled with mechanical positioning aids, which press the gloves forward to their respective removal openings. The shape of the liner bases and positioning aids is accordingly complex, which makes the cost of these objects not negligible. In addition, use of such liner and positioning bases makes an enlarged dispensing box necessary, further raising the costs for materials and transport of these very price-sensitive disposable gloves.

### SUMMARY

It was thus an object of the present teaching to design a package for non-sterile gloves of the aforementioned type in such a way that the gloves do not need to be repackaged before they are used and so that in removing the gloves from the package it is possible to avoid infection of critical palm and finger regions by the possibly contaminated hands of the user. This is intended to interrupt an important contamination chain for nosocomial infections and external glove contamination. The inventive package is intended for one-time use, and its external dimensions should not be greater than those of conventional dispensing boxes (having only one centrally located removal opening) and it should be producible and fillable at comparable costs and at the same time should comprise removal openings sufficiently large as to allow easy removal of the gloves from the dispensing box.

The present teaching addresses this objective in such a way that the gloves are placed in parallel arrangement in the dispensing packages, but the removal openings should be placed at the edge of the dispensing package and sized in such a way that the gloves can be grasped and withdrawn from the dispensing box only in the area of the gloves' cuff edges. As in patent US 2007215628 A1, there are two glove stacks, arranged at a 180° angle, in the dispensing box, which are oriented in such a way that the finger region of the first stack is situated on or close to the cuff region of the second stack. However, contrary to US 2007215628 A1, the gloves are not separated by rigid liner bases, but instead, according to the present teaching, a flexible liner film, or preferably short bags of flexible material, are used to separate the glove stacks. In using a liner or separating film, it should preferably be longer and/or wider than the bases of the conventional rectangular dispensing box, so that the glove parts situated close to the package edges remain securely covered. Contrary to US 2007215628, such a design makes it possible to use substantially more compact and thus more economical package sizes, since any mechanical positioning aids can be dispensed with and, thanks to the flexible liner films or separating bags, the two glove stacks can be pressed together in the course of the packaging process in such a way that the air between gloves is completely expelled, and thus the flexible film conforms to the curvatures of the external dimensions of the individual glove stacks. For this reason, the dimensions of the separating film with respect to the base or covering surface of the glove box must be increased in both directions, to ensure that no touching of finger and palm regions of the gloves can occur, despite the resulting uneven course of the separating surfaces between the two glove stacks. In using the inventive layout, in addition, with conventional dispensing box designs for 100 pieces per package, it is possible to dispense with any type of device which presses the glove cuffs in the direction of the respective dispensing opening, because in using the inventive separating film the packaging dimensions can be kept small enough that no removal obstacles occur owing to the absence of such devices as described, for instance, in US 2007215628 or DE 29616735. Usually it is necessary to equip the dispensing box with two opposite removal openings, so that one removal opening is provided for each inserted stack. In such case the removal openings can be placed either diagonally opposite or opposite on the same dispensing box surface. The design and embodiment of the removal openings can be substantially freely formed, but their shape and placement should be selected so that the gloves can be grasped only by the glove cuff, and before removal the finger and palm regions of the gloves remain covered by the dispensing box or separating film or separating sack and thus uncontaminated.

The inventive separation of the two glove stacks during the packaging process is achieved—as described—for example by means of the insertion of a thin, flexible plastic or paper film, which preferably is somewhat larger in dimension than the liner base of the dispensing box.

A more highly developed embodiment, which can be advantageous for many an automated packing process, is to use several dividing films simultaneously. In this case, according to the present teaching, the finger and palm regions of each glove stack are, at least on one side, separately enveloped or covered by such a film and inserted into the dispensing box. The preferred width of the individual separating films here should be greater than the width of the dispensing box, in order to securely separate the two stacks of gloves. The length of the individual separating films should be selected in such a way that at least the single-side covering of the finger and palm regions is assured, but preferably the finger regions of the stack in question should be enveloped by the film.

Another embodiment of the spatial separation of the two inventive stacks—based on the foregoing explanations—consists in using short bags open on one side, so that at least the palm and finger regions of a stack of gloves are separately protected by a flexible bag of synthetic foil or paper and the glove cuffs extend out to provide unobstructed removal from the sack. The inventive separating bag accordingly comprises, at least on one side, an opening through which the gloves can be inserted and removed. However, embodiments of the separating bag open on both sides are also possible and consistent with the present teaching, so that preferably the two open sides are contiguous with one another at right angles, which can afford great advantages in automatic packaging. The inventive separation bag has the advantage that the hygienically critical palm and finger regions are always perfectly covered and thus the removal openings on the dispensing box can be of considerable size, thus greatly facilitating hygienic removal of gloves, in comparison with other embodiments such as, for instance, in US 2007215628. In addition, by using the inventive separating bag in the packaging process, the previously counted glove stacks are very easily prepared and the oriented insertion of the glove stacks is extremely facilitated. As a result, conventional dispensing boxes of cardboard can be used, so that the only necessary design modification is the positioning of the perforated areas for the removal openings. Optionally, stacks of gloves that are protected by separation bags from contamination in the palm and finger regions can also be packed in dispensing boxes individually or immediately oriented, so that here too one single, correspondingly sized and positioned removal opening would be sufficient.

The advantages of the present teaching can be summarized as follows: Owing to the inventive placement and orientation of the removal openings in the dispensing boxes of flexible gloves, contamination and dirtying of gloves in the critical palm and finger regions in the course of removal is substantially reduced. Commercially available, cost-optimized dispensing box materials and dimensions can be adopted without modifications, so that substantially only the position, dimensions and number of removal openings are newly established. The additional inventive separation of the glove stacks situated in the package—preferably by insertion of a thin synthetic film or a flexible paper or carton layer or by other means such as bags open on one side—can be integrated into the existing packaging processes easily and without notable modifications concerning costs, investments or package dimensions. The inventive flexible configuration of the separating films or glove bags makes possible an ideal adjustment of the flexible gloves to the dimensions of the dispensing box and thus—possibly by dispensing with other positioning aids integrated into the dispensing box—permits absolutely small and cost-effective package sizes. The separating films or separating bags executed according to the present teaching can also be arranged in such a way that both removal openings can be placed opposite one another on the same dispensing box surface, avoiding the need to rotate the dispensing box after use of half of the gloves, and boxes can be mounted, for example, into many of the currently available dispensing box wall brackets. In addition, owing to the inventive flexible separation of the glove stacks, it remains possible to expel air from inside the gloves and to reduce the volume of the stack in the course of the packaging process by deliberate, preferably simultaneous pressure on both stacks, likewise contributing essentially to the maintenance of small and economical package dimensions and low shipping costs for the gloves.

Owing to the use of the inventive dispensing boxes for flexible medical gloves, a substantial contribution can be made toward reducing nosocomial infections in the health care sector. The use of inventive dispensing boxes for protective gloves of this kind is also relevant to many other sectors, such as for instance in cleanroom technology, biotechnology, microbiological laboratories and medical products, where reductions in contaminations can also lead to important cost savings and technological advantages.

As mentioned in the introduction to this application, the use of examination gloves can lead to increased perspiration accumulation and bacterial growth owing to the occlusive entrapment of human skin inside the glove, and in addition it can be assumed that gloved hands, even in the hospital and health care sector, in many cases, are not washed and disinfected often enough.

Therefore it is further proposed in the present teaching that the inventive dispensing boxes should be filled with disposable gloves that, in addition, contain one of the several germicidal or germ-growth-inhibiting substances on their interior and/or external surfaces.

Such surfaces are described, for example, in patent WO 2009/073907. The following usable disinfectants are accordingly proposed:

Chlorhexidine (not water-soluble)

Mecetronium ethyl sulphate (water-soluble)

Triclosan (not water-soluble)

Triclocarban (not water-soluble)

Octenidine (not water-soluble)

Chloramine T (water-soluble)

Thymol (not water-soluble)

Chloroxylenol (not water-soluble)

Chitosan (polymer, not water-soluble)

Polyhexanide (polymer, water-soluble)

Zinc, copper, silver salts

Among many others

The listed disinfectants are currently in use in health care institutions in the form of alcohol or aqueous solutions for disinfecting hands, skin and medical products. Judging from the many publications and guidelines for hospital hygiene now available, it appears likely that the use of inventive dispensing boxes, for instance for non-sterile disposable medical gloves, and the application of disinfectants to their surfaces, will lead to a drastic reduction in nosocomial infections.

At the same time, the use of gloves with surfaces having germicidal or germ-growth-inhibiting effects, combined with the inventive dispensing boxes, constitutes a new, synergistically effective method of combatting nosocomial infections.

### BRIEF DESCRIPTION OF THE DRAWINGS

FIG. 1a illustrates a stack of gloves.

FIG. 1b illustrates examination gloves placed in the dispensing boxes.

FIGS. 2a, 2b and 2c illustrate dispensing boxes.

FIG. 3 illustrates a dispensing case.

FIG. 4a illustrates a stack of gloves usable on either hand.

FIGS. 4b and 4c illustrate dispensing boxes filled with gloves.

FIG. 4d illustrates two removal openings 4a and 4b placed opposite one another on the same surface of the dispensing box.

FIGS. 5a and 5b illustrate two short separating films.

### DETAILED DESCRIPTION

Hereinafter, details of the present teaching are described in additional detail, with reference to the appended drawings:

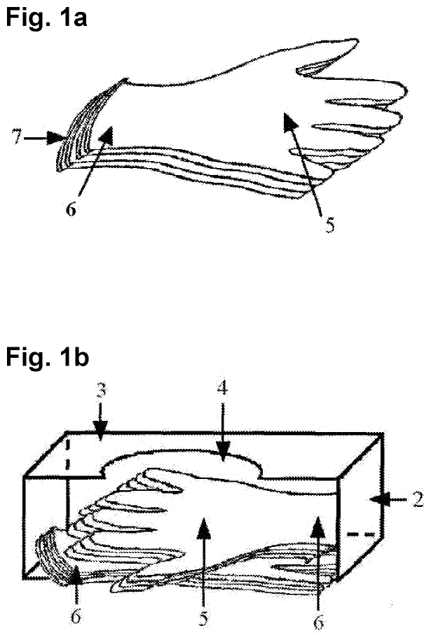

FIG. 1a shows a stack of gloves that can usually be used by either hand, for example examination gloves of latex, which according to the state of the art, are usually loaded in dispensing boxes. A non-sterile examination glove consists essentially of a hand portion (5), which contains the palms, backs of the hand and fingers, and a cuff part (6), which covers the wrist and possibly the forearm, and in most cases comprises a rolled edge (7) terminating the cuff.

FIG. 1b illustrates how examination gloves, according to the current state of the art, are placed in the dispensing boxes, and here, in the drawing, to make this clear, the front part of the dispensing box is not shown. The dispensing boxes each normally contain 100 gloves, with about 50 laid with the fingers facing left and the remainder with the fingers facing right, in a single stack. This arrangement has the advantage that the dispensing box is rather uniformly filled and, upon pressing together the gloves so arranged, air can be expelled well from the gloves, which allows very small dispensing box dimensions and can keep costs low for packing materials as well as storage and shipping. In addition, thanks to the careful stacking, removal of individual gloves through the removal opening (4) is appreciably facilitated. As can be seen clearly from FIG. 1b, in the removal process the glove is grasped by the user in the region of the palm (5) and pulled through the removal opening (4). Here, in practice, the result is always contaminations of the gloves in the critical palm region (5) by germs, usually along with contamination of adjacent gloves in the dispensing box.

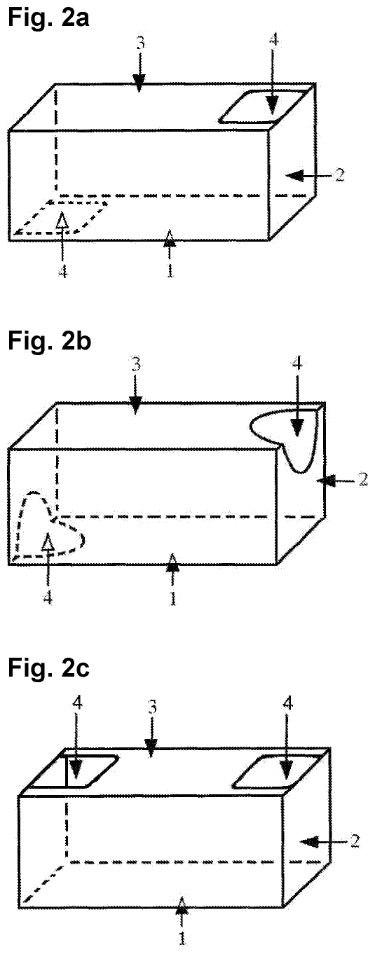

FIGS. 2a, 2b and 2c show dispensing boxes for the glove stacks, which are protected according to the present teaching by flexible paper films or separating bags, where, according to the present teaching, the two removal openings (4) are moved to the particular area of the package where the cuffs (6) or rolled edges (7) of the gloves are located. The inventive dispensing boxes here are preferably made of cardboard, so that the removal openings (4), conforming to the state of the art, can be produced by tearing out the corresponding pre-perforated surfaces or, in a different embodiment, can be re-closable.

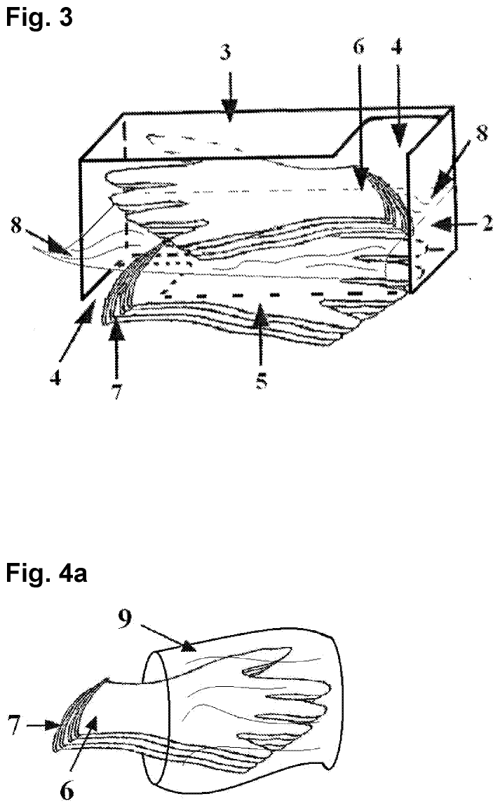

FIG. 3 shows such an inventive dispensing case, which is filled with gloves, with the front part of the dispensing box removed from the drawing for purposes of clarity. Owing to the staggered arrangement of the gloves, they can be grasped easily by the cuff (6) or rolled edge (7) and pulled in lengthwise direction out of the package. In the process, the hygienically critical regions in the palms and fingers (5) are kept constantly covered, and are protected from contaminations during removal.

The stacks of gloves here are separated by an inventive separating wall or separating film (8) consisting of plastic, paper, cardboard or other materials, such that the separating film is preferably larger than the base or covering surface of the dispensing box, so that the critical finger regions (5) can be somewhat enveloped and the gloves have a guarantee of contamination protection. Only by means of such an enlarged film surface can separation between the two stacks of gloves be assured. The separating film can be connected with the interior of the dispensing box as well as, only additionally, being inserted between the gloves during the process of packing them. The separating film (8) provides spatial separation and/or protection of the palm and finger regions (5) of the gloves situated under it before contact with the user's hand in the process of removal. In addition, the separating film (8) indicates the time when the user must switch to the other removal opening (4).

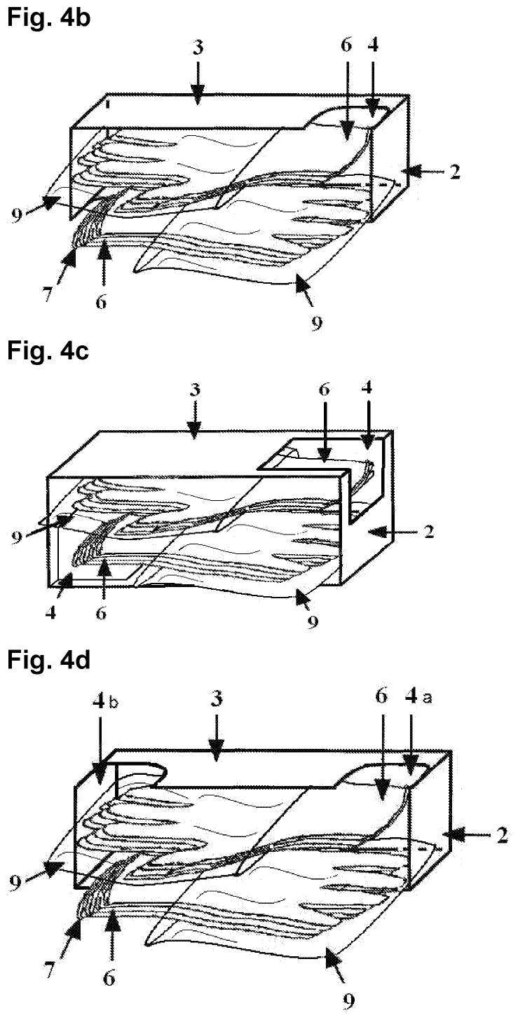

FIG. 4a shows a stack of gloves usable on either hand. Contrary to FIG. 1a, however, the palms and fingers of the glove, according to the present teaching, are protected here by a short bag (9), for example made of flexible polyethylene film material.

FIGS. 4b and 4c show, in a preferred embodiment, an inventive dispensing box filled with gloves, wherein for purposes of illustration the front portion of the dispensing box is not shown in the drawing. The variously oriented stacks of gloves situated in the dispensing box are visible here as well and are separated by means of inventive separating bags as described and shown in FIG. 4a. However, because of the bag-shaped design of the separating film (9)—as shown for example in FIG. 4c—the removal openings of the dispensing box can be much larger and thus have overlapping surfaces, which substantially facilitates removal of the gloves in comparison with other embodiments, such as in US 2007/215628, while at the same time the box dimensions can also be kept very small.

FIG. 4d shows the two removal openings 4a and 4b placed opposite one another on the same surface of the dispensing box, and here, as well, the front part of the dispensing box is not shown in the drawing. First the gloves of the upper stack of gloves are removed through the removal opening 4a, and then the upper separating bag is removed through one of the two removal openings 4a or 4b. At that point the gloves of the lower stack can be removed from the removal opening 4b, such that these as well remain protected from contamination in the finger and palm regions by the separate glove bag. Of course, an arrangement of the removal openings 4a and 4b on the same side of the dispensing box, even while extending beyond the surface analogously to FIG. 4c, is in accordance with the present teaching.

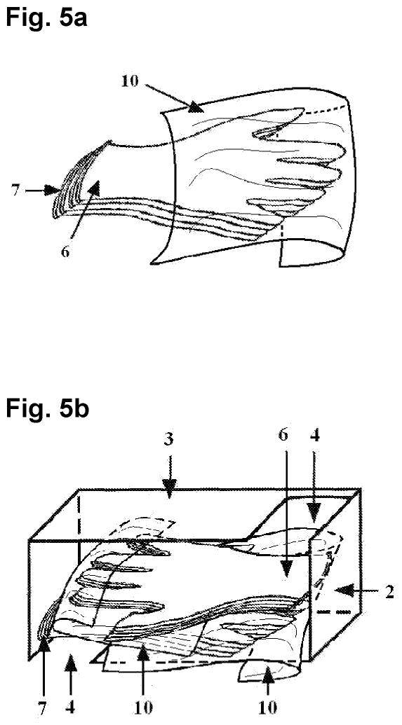

FIGS. 5a and 5b illustrate in one example the inventive use of two short separating films. Accordingly, as shown in FIG. 5a, the palm regions of the gloves are enveloped by the separating film, while the glove cuffs remain freely accessible, at least on one side. The two glove stacks equipped with short separating films, as drawn in FIG. 5b, are then inserted into the dispensing box. Here, once again, for purposes of clarity, the front part of the dispensing box is not included in the drawing.

1. A dispensing box for flexible gloves, has one or more removal openings, which are positioned in such a way that, when the gloves are arranged parallel in the dispensing box, the gloves can be grasped for removal only at the glove cuff, or at the cuff edge, or at a gripping aid positioned on the glove cuff, the dispensing box containing two or more stacks of gloves, oriented parallel at an 180° angle,
wherein the dispensing box contains in its interior no mechanical positioning aids for the gloves,
wherein, to protect the gloves from contamination in palm and finger regions, at least one separating film or a separating bag of flexible material is present in the dispensing box,
wherein the separating film or separating bag covers the finger and palm parts of at least one glove stack on at least one side.

2. The dispensing box for flexible gloves according to claim 1,
wherein the dispensing box contains at least two glove stacks, which are arranged parallel at an 180° angle, and comprises at least two removal openings,
wherein at least one separating film of flexible material is present in the dispensing box to protect the gloves from contamination in the palm and finger regions,
wherein the separating film covers the finger and palm parts of the substantially parallel oriented glove stacks on at least one side and is present in loose form in the preferred embodiment,
wherein the length and width of the separating film are greater than the length and width of the base or covering surface of the dispensing box, and
wherein the glove stacks are arranged in the dispensing box in such a way that they are completely separated by the separating film and do not touch one another.

3. The dispensing box for flexible gloves according to claim 1,
wherein the dispensing box contains at least two glove stacks, which are arranged at an 180° angle, and includes at least two removal openings situated opposite either lengthwise or diagonally,
wherein, to protect the gloves from contamination in the palm and finger regions, at least one flexible separating bag is mounted in the dispensing box, containing one glove stack, and
wherein the separating bag covers the finger and palm parts of the substantially parallel oriented glove stack and leaves the glove cuffs and/or the cuff edges easily accessible.

4. The dispensing box for flexible gloves according to claim 1,
wherein the dispensing box contains two glove stacks separate from one another, which are arranged parallel at a 180° angle, and includes at least two removal openings situated opposite either lengthwise or diagonally,
wherein each stack of gloves is separately equipped with a short film which covers the finger and palm regions, at least on one side,
wherein the width of the brief separating film is greater than the width of the base or covering surface of the dispensing box.

5. The dispensing box according to claim 1, wherein the flexible separating film or the flexible short separating film or the separating bag is made of paper or plastic.

6. The dispensing box according to claim 1, wherein the dispensing box comprises two diagonally opposite removal openings.

7. The dispensing box according to claim 1, wherein the dispensing box comprises two opposite removal openings on the same side of the dispensing box.

8. The dispensing box according to claim 1, wherein the outside surfaces of the dispensing box are made of cardboard.

9. The dispensing box according to claim 1, wherein at least one removal opening is obtained by tearing open a pre-perforated surface in the dispensing box.

10. The dispensing box according to claim 1, wherein the gloves have one of the several anti-germ or germ- growth-inhibiting substances on their inner and/or outer surfaces.
